(19) United States
(12) Patent Application Publication
(10) Pub. No.: US 2022/0193156 A1
(43) Pub. Date: Jun. 23, 2022

(54) PREOPERATIVE COMPOSITIONS AND METHODS OF PREPARATION THEREOF

(71) Applicant: Thi Tuyet Mai Ta, Kent, WA (US)

(72) Inventor: Thi Tuyet Mai Ta, Kent, WA (US)

(21) Appl. No.: 17/331,613

(22) Filed: May 26, 2021

Related U.S. Application Data
(60) Provisional application No. 63/130,348, filed on Dec. 23, 2020.

(51) Int. Cl. A61K 35/747; A61K 35/20; A61K 36/48; A61K 35/745; A61K 35/744; A61K 31/07; A61K 31/015; A61K 31/355; A61K 31/593; A61K 31/122; A61K 31/375; A61K 33/26; A61K 33/06; A61K 33/30; A61K 31/525; A61K 31/455; A61K 31/4415; A61K 31/4188; A61K 31/714; A61K 31/519; A61K 33/04; A61P 3/02; A61P 41/00; A61K 47/26; A61K 47/14

(52) U.S. Cl. CPC A61K 35/747; A61K 47/14; A61K 36/48; A61K 35/745; A61K 35/744; A61K 31/07; A61K 31/015; A61K 31/355; A61K 31/593; A61K 31/122; A61K 31/375; A61K 33/26; A61K 33/06; A61K 33/30; A61K 31/525; A61K 31/455; A61K 31/4415; A61K 31/4188; A61K 31/714; A61K 31/519; A61K 33/04; A61P 3/02; A61P 41/00; A61K 47/26; A61K 35/20

(57) ABSTRACT

Disclosed are preoperative compositions and methods of preparation thereof. The preoperative compositions may be for minimizing thirst, vomiting, insulin resistance, and the like prior to surgery. Such preoperative compositions may also reduce postoperative complications and the need for pain relievers, as well as minimize blood transfusion needed during and after surgery when administered the night before the operation and/or an hour before the operation. The preoperative compositions may include soymilk, isolate milk protein, isolate pea proteins, probiotics, whole milk powder, and micronutrients for example.

### CROSS-REFERENCE TO RELATED APPLICATION

This application claims the benefit of the filing date of U.S. Provisional Patent Application 63/130,348 entitled “PREOPERATIVE COMPOSITIONS AND METHODS OF PREPARATION THEREOF” to Ta Thi Tuyet Mai that was filed on Dec. 23, 2020, the disclosure of which is hereby incorporated herein by this reference.

### TECHNICAL FIELD

The present disclosure relates to preoperative compositions and methods of preparation thereof, and more specifically to preoperative compositions for administration to a patient the night before and an hour before surgery to both prepare for the medical procedure and to aid recovery from the procedure.

### BACKGROUND

Traditionally and for decades, patients have been mandated to fast the night before a surgery. The common fasting periods of 6 to 8 hours and sometimes as long as 10 to 12 hours aggravate insulin resistance, which is a marker of surgical stress. Currently, the American Anesthesia Association allows oral fluids up to 2 hours before surgery. On the other hand, the European Association for Clinical Metabolism and Nutrition recommends drinking sugar water two hours before administration of anesthesia. Unfortunately, these outdated practices persist despite emerging evidence revealing that excessive fasting results in negative outcomes and delayed recovery.

Many clinical studies have demonstrated preoperative carbohydrate loading can be reduce insulin resistance and increase postoperative patient satisfaction (for example, reducing thirst, nausea, and vomiting). Gastric contents that are considered to increase the risk of aspiration pneumonitis in anesthesia are a pH less than 2.5 and gastric volume of 0.4 ml/kg; but the preoperative dose of carbohydrates did not affect the preoperative gastric storage volume and pH, so giving carbohydrate beverages to the patients before surgery has become an essential step in the postoperative early recovery program (ERAS).

However, the current practice is not suitable for all patients. Most studies in the world have been performed on patients of European descent (in Europe and the United States), where patients were told to drink 800 ml of a solution containing 12.5% by weight the night before surgery and 400 ml of the same solution two hours before surgery. These volumes are relatively large compared to patients of Asian descent. For example, hospitals in Vietnam have not implemented the ERAS, because the anesthetist refuses to give local patients such a large volume of liquids before surgery. Accordingly, alternative solutions are needed where a smaller volume of preoperative solution could be administered without reducing its beneficial effects on insulin responsiveness, nausea and vomiting, and sensation of thirst.

### SUMMARY

Aspects of this document relate to preoperative compositions that improve patient outcomes and postoperative satisfaction when administered the evening before and in some implementations then at least an hour before surgery.

A preoperative composition may include a soy milk, isolate milk protein, isolate pea protein, a composition of probiotic organisms, and a composition of vitamins and minerals.

Implementations may include one or more or all of the following.

The composition of probiotic organisms may include at least one species of bacteria selected from the group consisting of: Lactobacillus acidophilus, Lactobacillus reuteri, Lactobacillus casei, Bifidobacterium longum, and Streptococcus faecalis. For example, the composition of probiotic organisms may include: L. acidophilus; L. reuteri; L casei; B. longum; and S. faecalis. The composition of probiotic organisms may be microencapsulated. The composition of probiotics may only be added into the nutritional composition prior to oral ingestion.

The composition of vitamins and minerals may include selenium and vitamin K2. The composition of vitamins and minerals may further include at least one vitamin or mineral selected from the group consisting of: retinol, carotene vitamin E, vitamin D, vitamin K1, a vitamin B, vitamin C, iron, magnesium, and zinc. Vitamin D may be vitamin D3. Vitamin B may be thiamin, riboflavin, niacin, vitamin B6, vitamin B7, vitamin B9, vitamin B12, or a combination thereof. The composition of vitamins and minerals may be microencapsulated.

The isolate milk protein may be at least partially provided by whole milk powder. The whole milk powder may be unsweetened. The isolate milk protein may be at least partially provided by demineralized whey powder.

The percent by weight of the isolate milk protein may be at least 3.3%.

The percent by weight of the whole milk powder may be between 8-9%.

The percent by weight of the isolate pea protein may be at least 2.4%.

The percent by weight of soy milk may be between 70-75%.

The preoperative composition may further include a medium chain triglyceride. The percent by weight of the medium chain triglyceride may be between 2-3%.

The preoperative composition may further include a sugar. The percent by weight of the sugar may be between 3-4%. The sugar may be fructose.

The preoperative composition may also include a sugar solution comprising 25% by weight sugar. The sugar provided by the sugar solution may be glucose, for example in the form of maltodextrin.

At least 20% of the protein content of the preoperative composition may be branched-chain amino acids.

Other aspects of this disclosure relate to methods of preparing a patient for surgery that may include administering to the patient the nutritional composition the evening before the surgery (e.g., at least six (6) hours prior to surgery), or may include administering to the patient the nutritional composition the evening before the surgery and then administering to the patient a sugar solution in the morning prior to surgery (e.g., at least one hour prior to the surgery or between an hour to three hours prior to the surgery). That way the patient ingests nothing in addition to the nutritional composition, the sugar solution, and water for at least six (6) hours prior to the surgery

Regardless of the particular method of preparing a patient for surgery, the nutritional composition may include: soy milk; isolate milk protein; isolate pea protein; a composition of probiotic organisms; and a composition of vitamins and minerals comprising selenium and vitamin K2.

Implementations may include one or more or all of the following.

The sugar solution may include 25% by weight maltodextrin.

The sugar solution may provide 50 g maltodextrin.

The sugar solution may provide between 0.5-1 mg sugar/kg body weight of the patient.

The nutritional composition may provide: 0.4±0.05 g isolate milk protein/kg body weight of the patient; 0.3±0.03 g isolate pea protein/kg body weight of the patient; 9.2±0.3 selenium/kg body weight of the patient; and 16.6±2.0 μg vitamin K2/kg body weight of the patient.

The nutritional composition may provide 0.2±0.02 g branched-chain amino acid/kg body weight of the patient.

The patient may be administered the nutritional composition between 6-12 hours prior to the surgery.

The patient may be administered the nutritional composition one evening before the surgery and the sugar solution at 6:00 AM on the surgery day.

The patient may be orally administered the nutritional composition and the sugar solution.

Still other aspects of this disclosure relate to methods of producing shelf-stable forms of the nutritional composition and the sugar solution.

The foregoing and other aspects, features, and advantages will be apparent to those of ordinary skill in the art from the specification, drawings, and the claims.

### BRIEF DESCRIPTION OF THE DRAWINGS

Implementations will hereinafter be described in conjunction with the appended drawings, where like designations denote like elements.

FIG. 1A depicts, in accordance with certain implementations, the percentage of patients in four groups who were determined by the Homeostasis Model Assessment to have insulin resistance before administration of anesthesia and two after stitched incision (percentage of patients, who had HOMA-IR≥2.5, before induction of anesthesia and 2 hours after stitched incision). Group 1 patients fasted prior to their surgery (“Fasting”). Group 2 patients drank only a placebo composition (aspartame 0.035%, 400 mL) the night before their surgery and another dose of the placebo composition (aspartame 0.035%, 200 mL) at 6:00 AM on their operation day (“Placebo”). Group 3 patients drank 400 ml of a composition comprising Maltodextrin 25% the night before their surgery followed by 200 ml of the same composition at 6:00 AM on their operation day (“CH Loading”). Group 4 patients drank the 300 ml of the disclosed nutritional composition the night before their surgery and 200 ml of the disclosed sugar solution at 6:00 AM on their operation day (“ONS Loading”). *p, significant difference levels between 4 groups were calculated by Chi-Square test (p*)

FIG. 1B depicts, in accordance with certain implementations, the percentage of patients in four groups who developed postoperative complication. Group 1 patients fasted prior to their surgery (“Fasting”). Group 2 patients drank only a placebo composition (aspartame 0.035%, 400 mL) the night before their surgery and another dose of the placebo composition (aspartame 0.035%, 200 mL) at 6:00 AM on their operation day (“Placebo”). Group 3 patients drank 400 ml of a composition comprising Maltodextrin 25% the night before their surgery followed by 200 ml of the same composition at 6:00 AM on their operation day (“CH Loading”). Group 4 patients drank the 300 ml of the disclosed nutritional composition the night before their surgery and 200 ml of the disclosed sugar solution at 6:00 AM on their operation day (“ONS Loading”).

FIG. 2 depicts, in accordance with certain implementations, a schematic for the production of the nutritional composition disclosed herein.

FIG. 3 depicts, in accordance with certain implementations, a schematic for the production of the sugar solution disclosed herein.

### DETAILED DESCRIPTION

While this disclosure includes a number of implementations that are described in many different forms, there is shown in the drawings and will herein be described in detail particular implementations with the understanding that the present disclosure is to be considered as an exemplification of the principles of the disclosed compositions and methods and is not intended to limit the broad aspect of the disclosed concepts to the implementations illustrated.

In the following description, reference is made to the accompanying drawings which form a part hereof, and which show by way of illustration possible implementations. It is to be understood that other implementations may be utilized, and component, as well as procedural, changes may be made without departing from the scope of the present disclosure. As a matter of convenience, various compositions and methods will be described using exemplary specifications, amounts, ranges, steps, procedures, and the like. However, this document is not limited to the stated examples and other compositions and methods are possible and within the teachings of the present disclosure. As will become apparent, changes may be made in the function, use, and/or arrangement of any of the components and steps described in the disclosed exemplary implementations without departing from the spirit and scope of this disclosure.

In the following description, and for the purposes of explanation, numerous specific details are set forth in order to provide a thorough understanding of the various aspects of the present disclosure. It will be understood, however, by those skilled in the relevant arts, that the present disclosure may be practiced without these specific details. It should be noted that there are many different and alternative compositions, methods, configurations, devices and technologies to which the present disclosure may be applied. The full scope of the present disclosure is not limited to the examples that are described below.

Described herein are nutritional compositions for administration the night before surgery and sugar solutions for administration at least one hour before surgery, where the administration of the compositions facilitates postoperative recovery in the patient, including recovery from general anesthesia. Also described is a method of preparing a patient for surgery comprising administering to the patient a nutritional composition described herein the night before the surgery and then administering to the patient a sugar solution described herein at least one hour before surgery, preferably no more than three hours before surgery, for example at two hours prior to surgery. In certain implementations, the sugar solution is administered to the patient at 6:00 AM on the day of surgery.

The administration of such compositions to the patient according to methods described herein reduces the patient's thirst, incidence of vomiting, insulin resistance, complications after surgery. As shown in the examples, the Comprehensive Complication Index of patients given the nutritional composition the night before the surgery and then the sugar solution two hours before surgery (referred to herein as “the intervention group”) was only 29.6±4.2 compared to a patient who just fasted before surgery (referred to herein as “the fasting group”), 45.2±4.5, p<0.05. The mean of postoperative morbidity survey of the intervention group was lower than that of the fasting group, 1.7±0.2 compared with 2.6±0.2, p<0.05.

In some aspects, the administration of such compositions also reduces pain reliever use and minimizes blood transfusion after surgery. As shown in the examples, the amount of blood transfused in the intervention group was only ⅙ of the fasting group (33±19 ml compared with 186±65 ml, p<0.05).

### 1. Terminology and Definitions

In describing implementations of preoperative compositions and methods of preparation thereof, the following terminology will be used in accordance with the definitions and explanations set out below. Notwithstanding, other terminology, definitions, and explanations may be found throughout this document, as well. Unless specifically noted, it is intended that the words and phrases in the specification and the claims be given their plain, ordinary, and accustomed meaning to those of ordinary skill in the applicable arts.

The singular forms “a,” “an,” and “the” include plural referents unless the context clearly dictates otherwise. Thus, for example, reference to “a step” includes reference to one or more of such steps.

The word “exemplary,” “example,” or various forms thereof are used herein to mean serving as an example, instance, or illustration. Any aspect or implementation described herein as “exemplary” or as an “example” is not necessarily to be construed as preferred or advantageous over other aspects or implementations. Furthermore, examples are provided solely for purposes of clarity and understanding and are not meant to limit or restrict the disclosed subject matter or relevant portions of this disclosure in any manner. It is to be appreciated that a myriad of additional or alternate examples of varying scope could have been presented, but have been omitted for purposes of brevity.

As used herein, the term “surgery” refers to a medical procedure performed for the purpose of structurally altering the human body by the incision or destruction of tissues where general anesthesia is required. As used herein, the term encompasses the diagnostic or therapeutic treatment of conditions or disease processes by any instruments causing localized alteration or transposition of live human tissue which include lasers, ultrasound, ionizing radiation, scalpels, probes, and needles. The tissue can be cut, burned, vaporized, frozen, sutured, probed, or manipulated by closed reductions for major dislocations or fractures, or otherwise altered by mechanical, thermal, light-based, electromagnetic, or chemical means. As referenced herein, the start of surgery is when the patient is given general anesthesia.

As used herein, the term “isolate milk protein” refers to a protein powder produced from milk, preferably from a ruminant animal, for example, a cow or a goat, which includes whey and casein. Accordingly, the isolate milk protein includes all nine essential amino acids. In some aspects, the isolate milk protein is free of lactose. In other aspects, the isolate milk protein includes at least one ingredient selected from whole milk powder, demineralized whey powder 40% (also referred to herein as “whey demin 40”), isolate whey and protein.

As used herein, the term “isolate pea protein” refers to protein isolated from peas, preferably the yellow pea. The isolate pea protein includes all nine essential amino acids and is a great source of branched-chain amino acids.

As used herein, the term “surgical stress” refers to a systemic response to surgical injury. It is characterized by activation of the sympathetic nervous system, endocrine responses, and immunological and histological changes, for example, perturbations in the inflammatory, acute, phase, hormonal, and genomic responses. In some circumstances, metabolism is impacted, and thus optimal nutrition support is often required to ensure positive patient outcomes following forgery. In some aspects, oxidative stress is also an indicator of surgical stress.

### 2. The Nutritional Composition

Some studies show that supplementation with branched-chain amino acids can reduce muscle mass loss, muscle contraction loss, and postoperative acute inflammatory response. Studies have also shown that the administration of probiotics one day before and for fifteen days after surgery helps reduce the incidence of complications after surgery, especially preventing severe infections. Additionally, low preoperative blood selenium level is also a risk factor for postoperative mortality. Reduced selenium concentration in the blood has been shown to be correlated to multiple organ failure. In fact, survivors of surgery have higher blood selenium levels than patients who died during surgery. Selenium acts as a co-enzyme in the endogenous defense system that protects cells from oxidative stress. A study found that four weeks of supplementing with 200 μg selenium before heart surgery improved insulin resistance. One preoperative nutrition guideline recommended providing 150-210 μg selenium dose to protect cells from oxidative stress. Vitamin K2 is not only a vitamin but a hormone that helps increase the toughness of the vessel wall and tissue integrity. A dose of 360 μg of vitamin K2 for 14 weeks was shown to increase arterial wall strength, improve renal tissue, and increase bone mass and was found not to be toxic. Thus, the nutritional composition described herein provides these needed nutrients for improved recovery from surgical stress. Accordingly, the nutritional composition includes branched-chain amino acids, selenium, and vitamin K2.

Both isolate milk protein and isolate pea protein contains high levels of branch chain amino acids. In a particular implementation, the nutritional composition includes soy milk, isolate milk protein, isolate pea protein, a composition of probiotic organisms, and a composition of vitamins and minerals comprising at least selenium and vitamin K12. The soy milk is prepared from 100 g of soybeans in 1000 ml water. In some aspects, one weight percent of the soymilk is protein. In some implementations, the isolate milk protein is at least partially produced by whole milk powder, preferably unsweetened whole milk powder. In some implementations, the nutritional composition includes between 3-5 g branched chain amino acids, for example, provided from 4-6 g isolate pea protein, the soy milk, and between 6-10 g isolate milk protein (which also originates from the unsweetened whole milk powder). In some aspects, the nutritional composition includes at least 3% by weight isolate milk protein and at least 2.4% by weight isolate pea protein.

The composition of probiotic organisms includes at least one species of bacteria selected from the group consisting of Lactobacillus acidophilus, Lactobacillus reuteri, Lactobacillus casei, Bifidobacterium longum, and Streptococcus faecalis. In some aspects, the composition of probiotic organisms is microencapsulated. In certain implementations, the composition of probiotic organisms includes 3×10⁸ colony-forming units of each species of bacteria.

The composition of vitamins and minerals includes between 0.5-1.0 g vitamin K2 and between 150 and 300 μg selenium. In some implementations, the composition of vitamins and minerals further includes at least vitamin or mineral selected from the group consisting of: retinol, carotene, vitamin E, vitamin D, vitamin K1, a vitamin B, vitamin C, iron, magnesium, and zinc. In certain implementations, vitamin D is vitamin D3. In some aspects, vitamin B includes thiamin, riboflavin, niacin, vitamin B6, vitamin B7, vitamin B9, vitamin B12, or a combination thereof. In particular implementations, the composition of vitamins and minerals includes 850-900 μg retinol, 4-7 mg carotene, 10-20 mg vitamin E, 10-20 mg vitamin D, 70-90 μg vitamin K1, 0.5-1.5 mg thiamin, 0.5-1.5 mg riboflavin, 10-20 mg niacin, 0.5-2.0 mg vitamin B6, 20-30 μg vitamin B7, 350-450 μg vitamin B9, 1.5-4.0 μg vitamin B12, 70-80 mg vitamin C, 5-10 mg iron, 300-350 mg magnesium, and/or 20-30 μg zinc.

In certain implementations, the nutritional composition also includes medium chain triglyceride. For example, the nutritional composition includes 2-3% by weight medium chain triglyceride. In some aspects, the nutritional composition also includes natural and artificial flavors, sweeteners, salt, flavor enhancers, color additives, emulsifiers, stabilizers, fats, and/or preservatives.

In a particular implementation, the nutritional composition includes:

between 70-75% by volume soymilk;

between 8-10% by weight whole milk powder;

between 2-4% by weight isolate milk protein; and

between 2-4% by weight isolate pea protein.

In a particular implementation, the ingredients of the nutritional composition and their respective amounts are listed in Table 3.

In some aspects, the nutritional composition is a 300 ml solution comprising between 15-20 g protein, of which at least 20% are branched-chain amino acids. For example, the nutritional the nutritional composition is a 300 ml solution containing 18 g protein, where 20.7% of the protein are branched-chain amino acids. The protein distribution of the nutritional composition is high in cysteine, which acts as an endogenous antioxidant.

In some implementations, the nutritional composition provides between 350-400 Kcal, between 15-20 g protein (of which at least 20% are branched-chain amino acids), 35-40 g carbohydrate, and 20-25 g fat. In particular implementations, the nutritional composition provides 390 Kcal in the form of 18 g protein, 39 g carbohydrate, and 21.6 g fat. In some implementations, the nutritional composition provides between 23.5 to 30 Kcal/kg body weight of the patient, 0.18-0.22 g branched-chain amino acid/kg body weight of the patient, 14-19 μg vitamin K2/kg body weight of the patient, and 8.5-10 μg selenium/kg body weight of the patient. Table 1 lists the nutritional information for certain implementations of the nutritional composition.

TABLE 1

| | | From | | | per kg |
|---|---|---|---|---|---|
| | | 300 ml | | | body weight |
| | | nutritional | Dietary | | from both |
| | | compo- | Reference | | preoperative |
| Nutrients | Units | sition | Intakes | | compositions |
| Energy | Kcal | 390.0 | 35 | Kcal/kg | 26.8 ± 3.2 |
| Protein | g | 18.0 | 1.5 | g/kg | 0.8 ± 0.1 |

| | | | | |
|---|---|---|---|---|
| Branched-chain amino | g | 3.7 | | 0.2 ± 0.02 |
| acids | | | | |
| Isolate milk protein | g | 8.7 | | 0.4 ± 0.05 |
| Isolate pea protein | g | 5.8 | | 0.3 ± 0.03 |
| Carbohydrate | g | 39.0 | 130 | 4.1 ± 0.5 |
| Fiber | g | 5.7 | 19-27 | 0.3 ± 0.03 |
| Lactose | g | 16.4 | 20 | 0.8 ± 0.1 |
| Glucose | g | 7.7 | | 0.4 ± 0.04 |
| Fat | g | 21.6 | 70-80 | 1.0 ± 0.1 |
| Transfat | g | 0.0 | 0.0 | 0.0 ± 0.0 |
| Mono Unsaturated | g | 1.7 | 29-34 | 0.1 ± 0.01 |
| Fatty Acid | | | | |
| Poly Unsaturated | g | 1.7 | 16-21 | 0.1 ± 0.01 |
| Fatty Acid | | | | |
| Saturated Fatty Acid | g | 9.4 | 20-30 | 0.4 ± 0.1 |
| Omega-3 | g | 0.1 | 2.2 | 0.004 ± 0.001 |
| Omega-6 | g | 2.1 | 14-18 | 0.1 ± 0.01 |
| Cholesterol | mg | 30.3 | <600 | 1.4 ± 0.2 |
| MCT | g | 6.3 | 52-103 | 0.3 ± 0.03 |

Vitamins

| | | | | |
|---|---|---|---|---|
| Vitamin A-Retinol | μg | 900.0 | 900-3000 | 41.5 ± 5.0 |
| Carotene | mg | 6.0 | 6-15 | 0.3 ± 0.03 |
| Vitamin E-Tocopherol | mg | 15.0 | 15-22 | 0.7 ± 0.1 |
| Vitamin D3-Calciferol | μg | 15.0 | 15-20 | 0.7 ± 0.1 |
| Vitamin K1 | μg | 75.0 | 75-120 | 3.5 ± 0.4 |
| Vitamin K2** | μg | 360.0 | 360 | 16.6 ± 2.0 |
| Vitamin B1-Thiamine | mg | 0.9 | 0.9-1.2 | 0.04 ± 0.01 |
| Vitamin B2-Riboflavin | mg | 0.9 | 0.9-1.3 | 0.04 ± 0.01 |
| Vitamin PP-B3-Niacin | mg | 14.0 | 14-16 | 0.6 ± 0.1 |
| Vitamin B5- | mg | 18.5 | 5 | 0.9 ± 0.1 |
| Pantothenic acid | | | | |
| Vitamin B6- | mg | 1.3 | 1.3-1.7 | 0.1 ± 0.01 |
| Pyridoxine | | | | |
| Vitamin B7-B8- | μg | 25.0 | 25-30 | 1.2 ± 0.1 |
| Vitamin H | | | | |
| Vitamin B9-Folate | μg | 400.0 | 400 | 18.5 ± 2.2 |
| Vitamin B12- | μg | 2.4 | 2.4 | 0.1 ± 0.01 |
| Cyanocobalamine | | | | |
| Vitamin C-Ascorbic | mg | 75.0 | 75-90 | 3.5 ± 0.4 |

Minerals and Trace Elements

| | | | | |
|---|---|---|---|---|
| Calcium | mg | 393.0 | 1000-1300 | 18.1 ± 2.2 |
| Phosphorus | mg | 462.0 | 700-1250 | 21.3 ± 2.6 |
| Iron | mg | 8.0 | 8-18 | 0.4 ± 0.04 |
| Sodium | mg | 177.0 | 1200-1500 | 8.2 ± 0.1 |
| Potassium | mg | 768.0 | 2000-4700 | 35.4 ± 4.3 |
| Magnesium | mg | 320.0 | 320-420 | 14.8 ± 1.8 |
| Zinc | mg | 25.0 | 25 | 1.2 ± 0.1 |
| Manganese | mg | 0.6 | 1.8-2.3 | 0.03 ± 0.004 |
| Copper | μg | 261.0 | 900 | 12.0 ± 1.5 |
| Fluoride | μg | 0.0 | 3000-4000 | 0.0 ± 0.0 |
| Iodine | μg | 6.7 | 150 | 0.3 ± 0.04 |
| Selenium | μg | 200.0 | 500 | 9.2 ± 0.3 |

### 3. The Sugar Solution

Between one and three hours prior to surgery, the patient is orally administered 200 ml of the sugar solution. In some aspects, the amount sugar provided to the patient via the sugar solution is 2 to 3 g sugar/kg body weight of the patient, for example, around 2.0 g/kg body weight, around 2.1 g/kg body weight, around 2.2 g/kg body weight, around 2.3 g/kg body weight, around 2.4 g/kg body weight, around 2.5 g/kg body weight, or around 2.6 g/kg body weight. In a particular implementation, the sugar solution includes 25% sugar in a water-based composition. The sugars provide glucose and may be maltodextrin. Table 2 lists the nutritional information for certain implementations of the sugar solution.

TABLE 2

| | | From | | Nutrients intake per |
|---|---|---|---|---|
| | | 200 ml | Dietary | kg body weight from |
| | | Sugar | Reference | both preoperative |
| Nutrients | Units | Solution | Intake | compositions |
| Energy | Kcal | 190 | 35 Kcal/kg | 26.8 ± 3.2 |
| Carbohydrate | g | 50 | 130 | 4.1 ± 0.5 |
| Maltodextrin | g | 50 | | 2.3 ± 0.3 |

### EXAMPLES

The present disclosure is further illustrated by the following examples that should not be construed as limiting. The contents of all references, patents, and published patent applications cited throughout this application, as well as the Figures, are incorporated herein by reference in their entirety for all purposes.

### 1. Method of Producing an Exemplary Nutritional Composition

Table 3 lists show the weight or volume percent distribution of the ingredients in an exemplary nutritional composition. Table 4 lists the ingredients by group for the production process. Table 5 lists the nutritional information for the exemplary nutritional composition.

TABLE 3

| | Percentage by | |
|---|---|---|
| Ingredient | Weight/Volume | |
| Soy milk 1% protein (1000 ml of soy milk made | 72.2% | (ml/100 ml) |
| from 100 g soybeans) | | |
| Unsweetened whole milk powder (24% protein) | 8.6% | (g/100 ml) |
| Milk Protein Isolate (88% protein) | 3.3% | (g/100 ml) |
| Pea Protein Isolate (80% protein) | 2.4% | (g/100 ml) |
| Water | 9.2% | (ml/100 ml) |
| Microencapsulation probiotics and other | 13.5% | (g/100 ml) |
| supplements: | | |
| Whey Demin 40; Nondairy-cream (Nondairy- | | |
| cream); MCT (Medium-chain Tryglyxerit); | | |
| Fructose Oligo Saccharic; Salt; | | |
| Palsgaard ®RecMilk (emulsifier and | | |
| stabilizer); Elemental iron (iron III | | |
| hydroxide); Magne; Elemental zinc (zinc | | |
| gluconate); Selenium; vitamin A (Retinyl | | |
| acetate); Carotene; vitamin E (DL-α | | |
| Tocopheryl acetate); vitamin D3; vitamin | | |
| K1; vitamin K2; vitamin B1-Thiamine; | | |
| vitamin B2-Riboflavin; Vitamin PP-B3- | | |
| Niacin; vitamin C-Ascorbic; vitamin | | |
| B6; vitamin B7; vitamin B9 (folic acid); | | |
| vitamin C. | | |

TABLE 4

| Group | Ingredients | Quantity | |
|---|---|---|---|
| 1 | Soy milk (1.1% protein) | 72.2% | (ml/100 ml) |
| 2 | Unsweetened whole milk powder | 8.6% | (g/100 ml) |
| | (24% protein) | | |
| 3 | Milk Protein Isolate | 3.3% | (g/100 ml) |
| | (88% protein) | | |
| 4 | Pea Protein Isolate | 2.4% | (g/100 ml) |
| | (80% protein) | | |
| 5 | Distilled water | 9.2% | (ml/100 ml) |
| 6 | Microencapsulated probiotics (in | | |
| | a simple microencapsulation sack) | | |
| | Lactobacillus acidophilus | 3 × 10⁸ CFUs/300 ml | |
| | Lactobacillus reuteri | 3 × 10⁸ CFUs/300 ml | |
| | Lactobacillus casei | 3 × 10⁸ CFUs/300 ml | |
| | Bifidobacterium longum | 3 × 10⁸ CFUs/300 ml | |
| | Streptococcus faecalis | 3 × 10⁸ CFUs/300 ml | |
| 7 | Supplements | | |
| | Whey demin 40 | 2.5% | (g/100 ml) |
| | Non dairy-cream | 3.5% | (g/100 ml) |
| | MCT (Medium chain | 2.5% | (g/100 ml) |
| | tryglyceride) | | |
| | Fructose Oligo Saccharide | 3.5% | (g/100 ml) |
| | Salt | 0.01% | (g/100 ml) |
| | Palsgaard ®RecMilk | 0.2% | (g/100 ml) |
| | (emulsifier and stabilizer) | | |

| | |
|---|---|
| Retinol | 3000 μg/1000 ml |
| Carotene | 20 mg/1000 ml |
| Vitamin E-Tocopherol | 48 mg/1000 ml |
| Vitamin D3 | 50 μg/1000 ml |
| Vitamin K1 | 224 μg/1000 ml |
| Vitamin K2 | 1200 μg/1000 ml |
| Vitamin B1-Thiamine | 3 mg/1000 ml |
| Vitamin B2-Riboflavin | 3 mg/1000 ml |
| Vitamin PP-B3-Niacin | 41 mg/1000 ml |
| Vitamin B6 | 4 mg/1000 ml |
| Vitamin B7 | 83 μg/1000 ml |
| Vitamin B9 | 1333 μg/1000 ml |
| Vitamin B12 | 5 μg/1000 ml |
| Vitamin C-Ascorbic | 250 mg/1000 ml |
| Iron | 21 mg/1000 ml |
| Magnesium | 819 mg/1000 ml |
| Zinc | 77 mg/1000 ml |
| Selenium | 667 μg/1000 ml |

TABLE 5

| | | | |
|---|---|---|---|
| | | Content/ | Content/ |
| Nutrients | Unit | 100 ml | 300 ml |

Main nutritional ingredients

| | | | |
|---|---|---|---|
| Energy | kcal | 130.0 | 390.0 |
| Protein | g | 6.0 | 18.0 |
| Branch Chain Amino Acid | % | 20.7 | 20.7 |
| Carbohydrate | g | 13.0 | 39.0 |
| Fiber | g | 1.9 | 5.7 |
| Lactose | g | 5.5 | 16.4 |
| Glucose | g | 2.6 | 7.7 |
| Fat | g | 7.2 | 21.6 |
| Transfat | g | 0.0 | 0.0 |
| Mono Unsaturated Fatty Acid | g | 0.6 | 1.7 |
| Poly Unsaturated Fatty Acid | g | 0.6 | 1.7 |
| Saturate Fatty Acid | g | 3.1 | 9.4 |
| Omega3 | g | 0.0 | 0.1 |
| Omega6 | g | 0.7 | 2.1 |
| Cholesterol | mg | 10.1 | 30.3 |
| MCT | g | 2.1 | 6.3 |

Vitamins

| | | | |
|---|---|---|---|
| Retinol | μg | 300.0 | 900.0 |
| Caroten | mg | 2.0 | 6.0 |
| Vitamin E-Tocopherol | mg | 5.0 | 15.0 |
| Vitamin D3 | μg | 5.0 | 15.0 |
| Vitamin K1 | μg | 25.0 | 75.0 |
| Vitamin K2 | μg | 120.0 | 360.0 |
| Vitamin B1-Thiamine | mg | 0.3 | 0.9 |
| Vitamin B2-Riboflavin | mg | 0.3 | 0.9 |
| Vitamin PP-B3-Niacin | mg | 4.7 | 14.0 |
| Vitamin B5 | mg | 6.2 | 18.5 |
| Vitamin B6 | mg | 0.4 | 1.3 |
| Vitamin B7 | μg | 8.3 | 25.0 |
| Vitamin B9 | μg | 133.3 | 400.0 |
| Vitamin B12 | μg | 0.8 | 2.4 |
| Vitamin C-Ascorbic | mg | 25.0 | 75.0 |

Minerals and trace elements

| | | | |
|---|---|---|---|
| Calcium | mg | 131.0 | 393.0 |
| Phosphorus | mg | 154.0 | 462.0 |
| Iron | mg | 2.7 | 8.0 |
| Sodium | mg | 59.0 | 177.0 |
| Potassium | mg | 256.0 | 768.0 |
| Magnesium | mg | 106.7 | 320.0 |
| Zinc | mg | 8.3 | 25.0 |
| Manganese | mg | 0.2 | 0.6 |
| Copper | μg | 87.0 | 261.0 |
| Fluoride | μg | 0.0 | 0.0 |
| Iodine | μg | 2.2 | 6.7 |
| Selenium | μg | 66.7 | 200.0 |
| Amino Acid Score* | | 978.9 | 978.9 |
| Inflammatory Index Score** | | 107.5 | 322.4 |

*WHO 2007. Protein and Amino Acid Requirements in Human Nutrition Report of A Joint WHO/FAO/UNU Expert Consultation. WHO, 935, 2007.
**Cavicchia et al. “A new dietary inflammatory index predicts interval changes in serum high-sensitivity C-reactive protein.” J Nutr. 2009; 139(12): 2365-2372.
≥200 Strong anti-inflammatory
101-200 Moderate anti-inflammatory
1-100 Mild anti-inflammatory
−1 to −100 Causes mild inflammation
−101 to −200 Moderate inflammation
≤−201 Causes strong inflammation

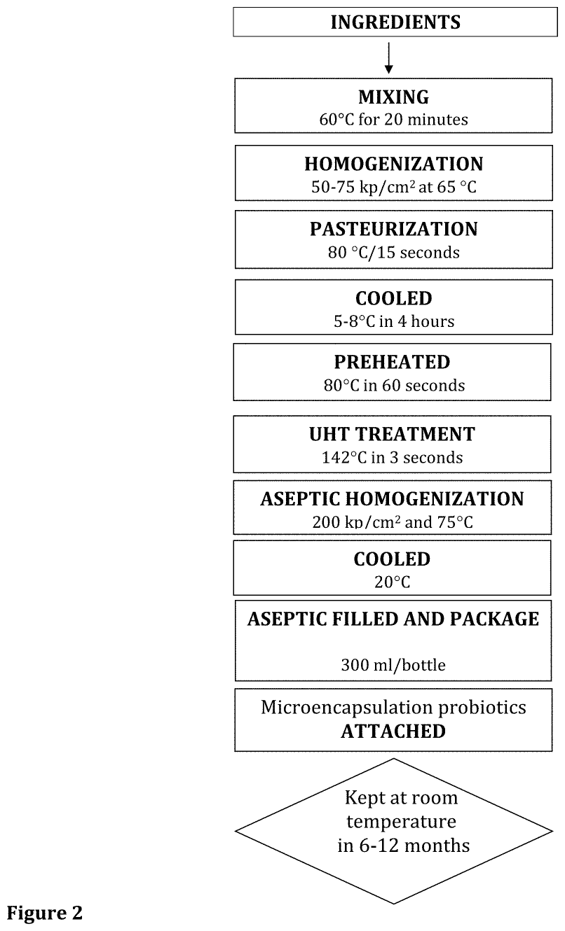

FIG. 2 is a schematic of the process of producing a nutritional composition described herein. The production process begins with heating water and soymilk 1% protein to a 50° C. temperature. Then the mixture of water and soymilk is mixed with unsweetened whole milk powder, whey demin 40, isolate whey protein, isolate pea protein, MCT, soluble fiber, and emulsifier by a mixing bath with a paddle stir. These ingredients are then heated at 60° C. for 20 minutes until the suspension is completely dissolved. The remaining nutrients are then added to the suspension, which is homogenized at 50-75 kp/cm² and 65° C.

The mixture is sterilized at 80° C. for 15 seconds by a thin panel or telescopic heat exchanger. Next, the suspension is cooled by a tube-type heat exchanger and held at 5-8° C. for 4 hours. The suspension is preheated at 80° C. for 60 seconds before undergoing ultra-high-temperature (UHT) process (142° C. for 3 seconds). The UHT-processed suspension is homogenized again in the aseptic condition at 200 kp/cm² and 75° C. The product is preserved by pouring into pasteurized packaging, 250 ml per unit, after cooled down to 20° C.

The final product may be kept at room temperature for at least six (6) months, though preferably less than 12 months. A composition of microencapsulated probiotics is attached to the product or designed to be contained in the lid of a milk carton. The composition microencapsulation probiotics includes L. Acidophilus 3×10⁸ CFU, L. Reuteri 3×10⁸ CFU, L. Casei 3×10⁸ CFU, B. Longum 3×10⁸ CFU, and S. Faecalis 3×10⁸ CFU.

### 2. Method of Producing an Exemplary Sugar Solution

Table 6 lists show the weight or volume percent distribution of the ingredients in an exemplary sugar solution. Table 7 lists the nutritional information for the exemplary nutritional composition.

TABLE 6

| Group | Ingredients | Quantity | |
|---|---|---|---|
| 1 | Maltodextrin | 25% | (25 g/100 ml) |
| | (type 10 Glucose molecules) 25% | | |
| 2 | Distilled water | 75% | (75 ml/100 ml) |

TABLE 7

| | | Content/ | Content/ |
|---|---|---|---|
| Nutrients | Measure | 100 ml | 200 ml |
| Energy | kcal | 95.0 | 190.0 |
| Carbohydrate | g | 25.0 | 50.0 |

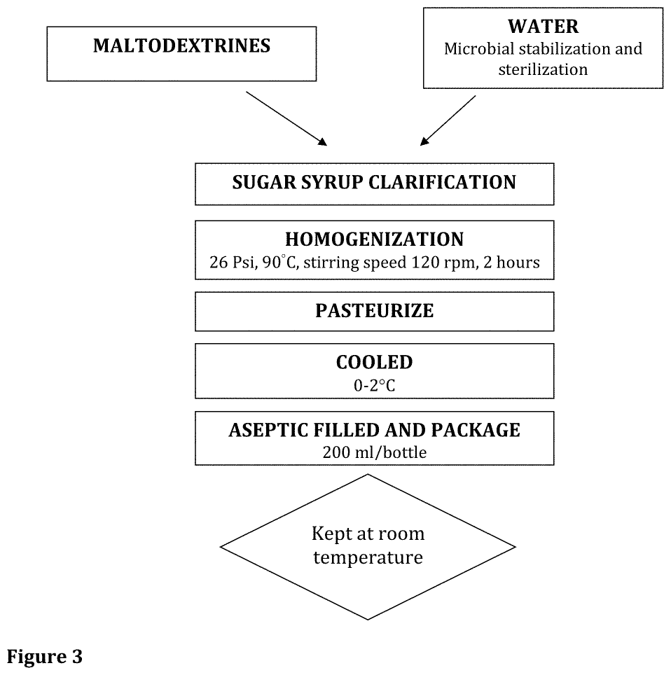

FIG. 3 is a schematic of the process of producing a sugar solution described herein. The process begins with pouring water into a cooking basin with an electric motor paddle and then adding maltodextrin to the bath so that the water content is five times the total sugar mass. The heated steam pressure was adjusted to 26 Psi within the cooking basin. The water and maltodextrin mixture is cooked with stirring (120 rpm) for about 2 hours until the solution reaches 90° C.—when bubbles appear and the mixture appears homogeneous.

The resulting syrup is removed from the cooking pot and filtered to remove impurities. The filtered syrup is them transferred to the 1500 liters tanks and cooled to 0-2° C. (for example, by the heat transfer pipes arranged in the tank's spiral). The cooled syrup is then pumped to the tank and transferred to the can extractor's extraction tank. The filling unit consists of 24 taps fitted with a valve system that automatically locks and opens, controlled by the POC programming system. Finished products are poured at the same time into 24 cans with a volume of 200 ml each.

Each can is then conveyed to the lid draw frame machine. All conveyors are controlled by a geared motor using 220 V power, capacity 0.5 Kw/h. After the lid is grafted, the conveyor belt will transfer the finished can to a water tank with four resistive bars to cool it up to a temperature of about 30° C. and moved to the shrink film packaging area and labeled.

### 3. Study of Impact on Metabolic Surgical Trauma, Hormonal Responses, Postoperative Complications, and Insulin Resistance

A randomized controlled clinical trial was conducted to evaluate whether drinking the described nutritional composition the night before surgery and the described sugar solution at 6:00 AM on the operation day could reduce metabolic surgical trauma, hormonal responses, postoperative complications, and insulin resistance.

144 ASA I-IV orthopedic patients were randomized into four groups, 36 patients in each group. Group 1 patients fasted prior to their surgery. Group 2 patients drank only a placebo composition (aspartame 0.035%, 400 mL) the night before their surgery and another dose of the placebo composition (aspartame 0.035%, 200 mL) at 6:00 AM on their operation day. Group 3 patients were part of the “carbohydrate loading” and drank 400 ml of a composition comprising Maltodextrin 25% the night before their surgery followed by 200 ml of the same composition at 6:00 AM on their operation day. Group 4 patients (named ONS) drank 300 ml of the nutritional composition the night before the surgery followed by 200 ml of the sugar solution.

Blood sample was collected immediately before the anesthesia induction, two hours after the stitched incision, and the fourth day after surgery. The samples were measured for blood glucose, plasma insulin, albumin, cholesterol, and CRP levels and lymphocyte count.

The patient's nutrition status was assessed on the day before operation and the 4th day after surgery. CCI, POMS, Epinephrine dose, and blood transfusion volume were collected. Nutritional status is estimated by NRS (Nutrition Risk Screening); the muscle mass of mid-upper arm (cm)13; Phage angle)(°; Extracellular Water/Total Body Water; Conut Score (Controlling Nutritional) which calculated from serum albumin (g/dl), lymphocytes/ml and cholesterol (mg/dl).

Gastric volume was assessed by ultrasound (a German LogiQe machine, at a frequency of 3.5 Mhz) before the induction of anesthesia.

The severity of disease requiring orthopedic surgery was assessed by AIS (Abbreviated Injury Scale). The physical status before surgery was measured by ASA. The risk of death from surgery is evaluated by a Preoperative Score to Predict Postoperative Mortality (POSPOM) and Prognostic Nutritional Index (PNI).

Pre-anesthesia thirst was assessed by a Likert scale.

Insulin resistance was determined by the Homeostasis Model Assessment (Homa-IR).

Postoperative complications were estimated by CCI (Comprehensive Complication Index), POMS (Postoperative morbidity survey), Epinephrine dose (mg), blood transfusion (ml), change of CRP (mg/l).

### Results

Table 8 shows the status of the patients at the beginning of the study. Table 9 lists the patient's status at the end of the study. Group 1 were the fasting group. Group 2 were the placebo group. Group 3 were the CH loading group. Group 4 were the ONS loading group.

In the beginning, most of the four groups' parameters were the same except for the severity of orthopedic surgery and the nutritional status. These placebo group's parameters were lower than that of the fasting and ONS loading group (Table 8).

TABLE 8

| | Group 1 | Group 2 | Group 3 | Group 4 |
|---|---|---|---|---|
| Variables | N = 36 | N = 36 | N = 36 | N = 36 |
| Age (years) | 58.8 ± 2.9 | 57.6 ± 2.9 | 57.2 ± 3.4 | 61.9 ± 3.3 |
| Disease status | | | | |
| Abbreviated Injury Scale | 2.7 ± 0.1^{a} | 3.0 ± 0.1^{b} | 2.9 ± 0.1^{ab} | 2.7 ± 0.1^{a} |
| Preoperative status | | | | |
| POSPOM5 | 11.0 ± 0.5 | 11.6 ± 0.4 | 11.5 ± 0.6 | 12.1 ± 0.6 |
| Likert scale of thirsty* | 2.1 ± 0.2^{a} | 1.8 ± 0.2^{ab} | 1.5 ± 0.1^{b} | 1.5 ± 0.1^{b} |
| Residual gastric volume*(ml) | 25.2 ± 4.6 | 29.4 ± 4.6 | 27.4 ± 4.0 | 20.9 ± 3.9 |
| Homa-IR⁶ | 3.7 ± 0.6^{ab} | 2.4 ± 0.3^{a} | 4.3 ± 0.8^{b} | 3.3 ± 0.5^{ab} |
| Prognostic Nutritional Index | 48.6 ± 1.0 | 49.3 ± 1.2 | 48.1 ± 1.2 | 50.6 ± 1.5 |
| CRP (C Reactive Protein) (mg/l) | 14.6 ± 3.8 | 15.7 ± 3.6 | 26.4 ± 7.3 | 23.9 ± 4.6 |
| Mean operative time (hours) | 1.7 ± 0.1 | 1.8 ± 0.1 | 1.7 ± 0.1 | 1.6 ± 0.1 |
| Blood loss (ml) | 234 ± 45 | 269 ± 61 | 205 ± 29 | 195 ± 26 |
| Nutritional status | | | | |
| Nutrition Risk Screening | 2.2 ± 0.3^{ab} | 2.8 ± 0.3^{a} | 2.2 ± 0.3^{ab} | 1.9 ± 0.3^{b} |
| Muscle mass of mid-upper arm | 18.8 ± 0.4 | 18.5 ± 0.4 | 19.0 ± 0.3 | 19.1 ± 0.5 |
| (cm) | | | | |
| Muscle strength (kg) | 21.9 ± 1.8 | 21.2 ± 1.9 | 25.1 ± 2.1 | 21.1 ± 1.9 |
| Phage angle (°) | 4.7 ± 0.2 | 4.6 ± 0.2 | 4.9 ± 0.2 | 4.8 ± 0.2 |
| ECW/TBW⁷ | 0.40 ± 0.004 | 0.41 ± 0.005 | 0.41 ± 0.005 | 0.40 ± 0.003 |
| Skeletal Muscle Index) (kg/m² | 7.2 ± 0.9 | 6.6 ± 0.6 | 8.1 ± 0.8 | 6.8 ± 0.6 |
| Conut Score ** | 1.7 ± 0.3 | 1.7 ± 0.3 | 1.9 ± 0.3 | 1.6 ± 0.3 |
| Serum Albumin (g/dl) | 38.2 ± 0.7 | 38.8 ± 0.9 | 37.9 ± 0.6 | 38.7 ± 0.8 |
| Lymphocytes/ml | 2094 ± 107 | 2100 ± 122 | 2036 ± 143 | 2396 ± 222 |
| Cholesterol (mg/dl) | 4.5 ± 0.2 | 4.5 ± 1.2 | 4.2 ± 0.2 | 4.4 ± 0.2 |

Means ± SEM.
⁵Preoperative Score to Predict Postoperative Mortality
⁶Homeostasis Model Assessment
⁷Extracellar Water/Total Body Water
*Before anesthesia
** Calculating from Serum Albumin (g/dl), Lymphocytes/ml and Cholesterol (mg/dl)
Values not sharing a common superscript letter in the same row are significantly different at p < 0.05. The difference in these data among four groups was analyzed by the Post hoc test following one-way ANOVA.

TABLE 9

| | Group 1 | Group 2 | Group 3 | Group 4 |
|---|---|---|---|---|
| Variables | N = 36 | N = 36 | N = 36 | N = 36 |
| Length of hospital stay (days) | 5.7 ± 0.6 | 5.4 ± 0.3 | 5.7 ± 0.5 | 5.2 ± 0.3 |
| Hospital fee (USD) | 751 ± 57 | 739 ± 57 | 722 ± 55 | 639 ± 29 |
| Digestive system | | | | |
| Likert scale of thirsty* | 2.1 ± 0.2^{a} | 1.8 ± 0.2^{ab} | 1.5 ± 0.1^{b} | 1.5 ± 0.1^{b} |
| Residual gastric volume*(ml) | 25.2 ± 4.6 | 29.4 ± 4.6 | 27.4 ± 4.0 | 20.9 ± 3.9 |
| Insulin resistance** | | | | |
| Change of Homa-IR⁵ | 2.03 ± 1.3^{a} | −0.23 ± 0.3^{ab} | −0.9 ± 0.9^{b} | 0.8 ± 0.7^{ab} |
| Complications | | | | |
| Comprehensive Complication | 45.2 ± 4.5^{a} | 39.7 ± 3.6^{ab} | 38.5 ± 4.4^{ab} | 29.6 ± 4.2^{b} |
| Index | | | | |
| Postoperative morbidity survey | 2.6 ± 0.2^{a} | 2.5 ± 0.2^{a} | 2.2 ± 0.2^{ab} | 1.7 ± 0.2^{b} |
| Epinephrine dose (mg) | 5.4 ± 1.4 | 2.8 ± 0.8 | 2.3 ± 1.0 | 5.1 ± 1.4 |
| Blood transfusion (ml) | 186 ± 65^{a} | 125 ± 42^{ab} | 65 ± 32^{b} | 33 ± 19^{b} |
| Change of CRP (mg/l) | −2.1 ± 1.2 | 0.9 ± 0.7 | −1.2 ± 1.1 | −0.2 ± 2.0 |
| Change of nutritional status*** | | | | |
| Nutrition Risk Screening | 0.2 ± 0.2 | 0.1 ± 0.2 | 0.4 ± 0.1 | 0.7 ± 0.2 |
| Muscle mass of mid-upper arm | 0.5 ± 0.4 | −0.2 ± 0.2 | −0.3 ± 0.3 | 0.1 ± 0.2 |
| (cm) | | | | |
| Muscle strength (kg) | −0.4 ± 1.0 | 0.0 ± 0.5 | 0.7 ± 0.7 | −0.5 ± 0.8 |
| Phage angle (°) | −0.32 ± 0.08 | −0.38 ± 0.05 | −0.32 ± 0.07 | −0.23 ± 0.09 |
| ECW/TBW ⁶ | 0.00 ± 0.00 | 0.00 ± 0.00 | −0.01 ± 0.00 | 0.01 ± 0.00 |
| Skeletal Muscle Index (kg/m²) | −0.58 ± 0.9 | −0.28 ± 0.6 | −1.76 ± 0.7 | −0.59 ± 0.4 |
| Conut Score | 1.5 ± 0.4 | 1.7 ± 0.3 | 1.6 ± 0.3 | 1.3 ± 0.3 |
| Serum Albumin (g/dl) | −4.1 ± 0.7 | −5.8 ± 0.7 | −4.2 ± 0.5 | −4.2 ± 0.7 |
| Lymphocytes/ml | −244 ± 134^{ab} | −187 ± 134^{ab} | −58 ± 159^{a} | −505 ± 207^{b} |
| Cholesterol (mg/dl) | −0.42 ± 0.1^{ab} | −0.66 ± 0.1^{a} | −0.35 ± 0.09^{b} | −0.44 ± 0.1^{ab} |

Means ± SEM.
⁵Homeostasis Model Assessment,
⁶ Extracellar Water/Total Body Water
*Before anesthesia,
**2 hours after completing operation,
***4 days after completing operation
Values not sharing a common superscript letter in the same row are significantly different at p < 0.05. The difference in these data among the four groups was analyzed by the Post hoc test following one-way ANOVA.

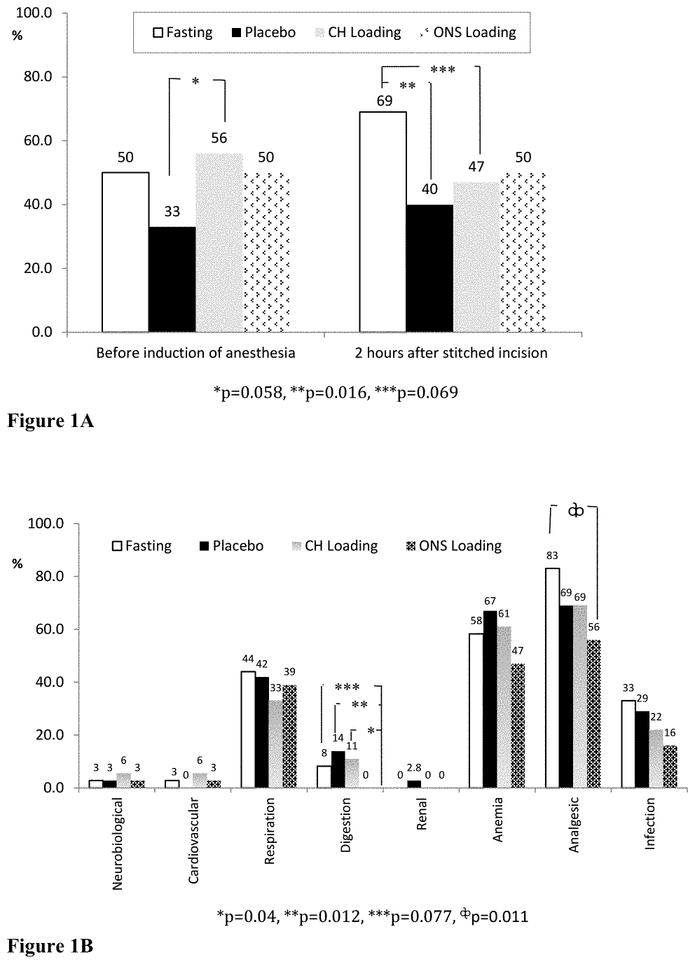

Regarding the efficacy of the intervention on the gastrointestinal tract, the fasting group had significantly more thirst than the CH loading group and ONS loading group. However, the gastric fluid volumes of the four groups were the same (Table 9). Equally important is the effect of the intervention on insulin resistance. Change of Homa-IR of CH loading Group, −0.9±0.9, was significantly lower than that of the fasting group, 2.03±1.3, p=0.026 (Table 9). The percentage of patients, who had HOMA-IR higher than 2.5 at 2 hours after stitched incision, came from the fasting group, which was significantly higher than that from the placebo group, 69% vs. 40%, p=0.016 (FIG. 1A).

Reducing postoperative complications is the most important effect of the intervention. CCI of the ONS loading group was significantly lower than that of the fasting group, 29.6±4.2. and 45.2±4.5, respectively, p=0.01 (Table 9). Moreover, POMS of the ONS group was significantly lower than that of both fasting group, 1.7±0.2 vs. 2.6±0.2, p=0.003, and of the placebo group, 1.7±0.2 vs. 2.5±0.2, p=0.013 (Table 9). The volume of blood transfusion of ONS loading group and CH loading group was significantly lower than that of the fasting group, 33±19 vs. 186±65, p=0.013 and 65±32 vs. 186±65, p=0.049, respectively (Table 9). The frequency of patients requiring additional painkillers after surgery in the fasting group was statistically significantly higher than that of the ONS group, 84% compared to 56% (FIG. 1B). While the ONS group had no patients with postoperative gastrointestinal complications, the placebo group had 14%, the CH loading group 11%, and the fasting group 8%, the difference was statistically significant (FIG. 1B). Lastly, ONS, placebo, or CH loading did not significantly affect postoperative orthopedic surgery patients' nutrition status (Table 9). It was the same for residual gastric volume (Table 9).

The result suggested that preoperative oral administration of either a sugar solution or the combination of the nutritional composition and the sugar solution reduced the development of insulin resistance in patients undergoing orthopedic surgery. Both preoperative treatments reduced the incidence of postoperative complications, but the combination of the nutritional composition and the sugar solution was especially effective.

In places where the description above refers to particular implementations, it should be readily apparent that a number of modifications may be made without departing from the spirit thereof and that these implementations may be alternatively applied. The accompanying claims are intended to cover such modifications as would fall within the true spirit and scope of the disclosure set forth in this document. The presently disclosed implementations are, therefore, to be considered in all respects as illustrative and not restrictive, the scope of the disclosure being indicated by the appended claims rather than the foregoing description. All changes that come within the meaning of and range of equivalency of the claims are intended to be embraced therein.

What is claimed:

1. A preoperative composition comprising:
soy milk;
isolate milk protein;
isolate pea protein;
a composition of probiotic organisms; and
a composition of vitamins and minerals comprising selenium and vitamin K2.

2. The preoperative composition of claim 1, wherein the composition of probiotic organisms comprises at least one species of bacteria selected from the group consisting of: Lactobacillus acidophilus, Lactobacillus reuteri, Lactobacillus casei, Bifidobacterium longum, and Streptococcus faecalis.

3. The preoperative composition of claim 2, wherein the composition of probiotic organisms comprises: L. acidophilus; L. reuteri; L casei; B. longum; and S. faecalis.

4. The preoperative composition of claim 1, wherein the composition of probiotic organisms is microencapsulated.

5. The preoperative composition of claim 1, wherein the composition of vitamins and minerals further comprises at least one vitamin or mineral selected from the group consisting of: retinol, carotene vitamin E, vitamin D, vitamin K1, a vitamin B, vitamin C, iron, magnesium, and zinc.

6. The preoperative composition of claim 5, wherein vitamin D is vitamin D3.

7. The preoperative composition of claim 5, wherein vitamin B is thiamin, riboflavin, niacin, vitamin B6, vitamin B7, vitamin B9, vitamin B12, or a combination thereof.

8. The preoperative composition of claim 1, wherein the composition of vitamins and minerals is microencapsulated.

9. The preoperative composition of claim 1, wherein the isolate milk protein is at least partially provided by whole milk powder.

10. The preoperative composition of claim 1, wherein the isolate milk protein is at least partially provided by demineralized whey powder.

11. The preoperative composition of claim 1, wherein:
the percent by weight of the isolate milk protein is at least 3.3%;
the percent by weight of the isolate pea protein is at least 2.4%; and
the percent by weight of soy milk is between 70-75%.

12. The preoperative composition of claim 1 further comprising a medium chain triglyceride.

13. The preoperative composition of claim 1 further comprising a sugar.

14. The preoperative composition of claim 13, wherein the percent by weight of the sugar is 3-4% and the sugar is fructose.

15. The preoperative composition of claim 1, wherein at least 20% of the protein content of the composition are branched-chain amino acids.

16. A method of preparing a patient for surgery, the method comprising:
administering to the patient a nutritional composition at least six (6) hours prior to surgery, wherein the nutritional composition comprises:
soy milk;
isolate milk protein;
isolate pea protein;
a composition of probiotic organisms; and
a composition of vitamins and minerals comprising selenium and vitamin K2; and
administering to the patient a sugar solution between one (1) hour and three (3) hours prior to surgery,
wherein the patient ingests nothing in addition to the nutritional composition, the sugar solution, and water for at least six (6) hours prior to the surgery.

17. The method of claim 16, wherein the sugar solution comprises 25% by weight maltodextrin.

18. The method of claim 16, wherein the sugar solution provides between 0.5-1 mg sugar/kg body weight of the patient.

19. The method of claim 16, wherein the nutritional composition provides:
0.4±0.05 g isolate milk protein/kg body weight of the patient;
0.3±0.03 g isolate pea protein/kg body weight of the patient;
9.2±0.3 μg selenium/kg body weight of the patient; and
16.6±2.0 μg vitamin K2/kg body weight of the patient.

20. The method of claim 16, wherein the nutritional composition provides 0.2±0.02 g branched-chain amino acid/kg body weight of the patient.
